(19) United States
(12) Patent Application Publication
(10) Pub. No.: US 2022/0039990 A1
(43) Pub. Date: Feb. 10, 2022

(54) Cervical Spine Rehabilitation Adjustment Device

(71) Applicant: New Century Products Limited, Taipei City (TW)

(72) Inventor: Fu-Tsun Lin, Taipei City (TW)

(21) Appl. No.: 16/985,235

(22) Filed: Aug. 5, 2020

(51) Int. Cl. A61F 5/02; A61F 5/055

(52) U.S. Cl. CPC A61F 5/026; A61B 5/4561; A61F 5/055

(57) ABSTRACT

The present invention provides a vertebrae adjustment device comprising a weight pocket, fixing rings, connection rings, a first fixing belt and a second fixing belt. The weight pocket is divided into a plurality of separations by connecting parts, and weight blocks are loaded into the plurality of separations. The first fixing belt has two first fixing sections and a through hole; wherein the two first fixing sections are respectively disposed at the front ends of the two bifurcated extensions of the first fixing belt, and fixed on the two first fixing rings respectively, and the through hole is provided on the rear end of the first fixing belt. The second fixing belt has two second fixing sections respectively disposed on both ends of the second fixing belt, and the second fixing belt passes through the through hole and is fixed on the two connecting rings.

### TECHNICAL FIELD OF THE INVENTION

The present invention relates to a cervical spine rehabilitation adjustment device, and more particularly to a cervical spine rehabilitation adjustment device capable of adjusting the number and weight of weight blocks to correct cervical vertebrae.

### DESCRIPTION OF THE PRIOR ART

With the advancement of technology, modern people's lifestyle has been changed by notebooks, tablet computers and smartphones; therefore, the cervical vertebrae disease affects daily life. Modern people often work long hours on their computers on the same sitting posture, or tilt their heads down to use their smartphones for a long time. Such postures cause their heads to lean forward more. After a busy day at work, it is often found that people experience a pain in the neck due to long periods of inactivity while maintaining the same posture. Using the computer and smartphone for a long time in wrong posture easily cause cervical vertebrae misalignment and cause cervical vertebrae pain. The cervical spine carries an estimated 5 kg of weight. If the head is tilted forward, the weight on the cervical vertebrae could increase to up to 13 to 18 kg, causing the cervical vertebrae to be prone to misalignment. The misalignment of the cervical vertebrae will not only cause neck and head pain, but also dizziness, tinnitus, or insomnia. If the hand nerves are compressed by the misaligned vertebrae, it will also cause shoulder pain, arm muscle pain, or a numbing sensation on the fingers.

In view of this, the inventor has invested a lot of research, development and effort, making breakthroughs and innovations, hoping to solve the current shortcomings with novel technical methods, not only bringing better products to the society, but also promoting industrial development at the same time.

### SUMMARY OF THE INVENTION

The main purpose of the present invention is to provide a vertebrae adjustment device by using weight blocks to adjust the problem of cervical vertebrae misalignment, so as to effectively improve the problems of pain in the arm muscle, shoulder, head, and neck, or finger numbness due to the compression of the hand nerves.

To achieve the above objective, the present invention provides a cervical spine rehabilitation adjustment device comprising a weight pocket, two fixing rings, two connection rings, a first fixing belt, a second fixing belt and a sensing device; wherein the weight pocket has a front plate, a rear plate, a bottom plate, a right side plate, a left side plate and a plurality of connecting parts. The front plate and the rear plate are connected to each other by the right side plate, the left side plate and the bottom plate to form an accommodating space; and the connecting parts are mounted in the accommodating space to divide the accommodating space into a plurality of separations. Further, the two fixing rings are mounted on the rear plate; and the two connection rings are respectively mounted on the right side plate and the left side plate. Furthermore, the first fixing belt has two first fixing sections and a through hole; wherein the two first fixing sections are respectively disposed on the front ends of the two bifurcated extensions of the first fixing belt, and the two bifurcated extensions of the first fixing belt are fixed on the two first fixing rings respectively by the two first fixing sections, and the through hole is provided on the rear end of the first fixing belt. The second fixing belt has two second fixing sections respectively disposed on both ends of the second fixing belt, and the second fixing belt passes through the through hole and is fixed on the two connecting rings by the two second fixing sections. The sensor is movably disposed on the vertebrae adjustment device through a fastener, and the sensor comprises a sensing unit and a processing unit electrically connected to the sensing unit; wherein the sensor senses movements of the head, neck or spine of a human body, and produces a sensing data indicating the activity state of the human body. The processing unit then generates a posture data of a cervical angle or a spine angle of the human body according to the sensing data, and produces a warning notification according to the posture data. In addition, the sensor is an inertial measurement unit (IMU), which is composed of three-axis gyroscopes and three-directional accelerometers.

In some embodiments, the cervical spine rehabilitation adjustment device further comprises a plurality of weight blocks loaded into the separations.

In some embodiments, the cervical spine rehabilitation adjustment device further comprises a plurality of notches disposed on the upper edge of the rear plate; and the weight blocks can be easily taken out or put in the separations through the notches.

In some embodiments, the two first fixing sections and the two second fixing section may be respectively a fastener or a velcro; wherein the two first fixing sections and the two second fixing section are respectively composed of a male fastener and a female fastener, and the male fastener is correspondingly fastened to the female fastener. Alternatively, the first fixing sections and the two second fixing section are respectively composed of a sticky part and a bonding part corresponding to the sticky part, and the sticky part is correspondingly bonded to the bonding part.

In some embodiments, the male fastener and the female fastener are sequentially arranged on the front ends of the first fixing belt and the second fixing belt, with a distance between the male fastener and the female fastener.

In some embodiments, the first fixing belt is composed of a first branch belt, a second branch belt, and a main body belt. The two first fixing sections are respectively disposed on the front ends of the first branch belt and the second branch belt, the through hole is provided on the rear end of the main body belt. The rear end of the first branch belt and the rear end of the second branch belt are connected to the front end of the main body belt, forming a ‘Y’ or ‘T’-shaped structure.

In some embodiments, the first branch belt and the second branch belt form a first included angle, and the angle is 30˜180 degrees.

In some embodiments, the first branch belt and the main body belt form a second included angle, the second branch belt and the main body belt form a third included angle, and the second included angle is equal to the third included angle.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a stereoscopical schematic view of a cervical spine rehabilitation adjustment device according to an embodiment of the present invention;

FIG. 2 is a top view of the cervical spine rehabilitation adjustment device according to the embodiment of the present invention;

FIG. 3 is a rear view of the cervical spine rehabilitation device according to the embodiment of the present invention;

FIG. 4 is a schematic view of the cervical spine rehabilitation adjustment device when worn, according to the embodiment of the present invention;

FIG. 5 is a side view of the cervical spine rehabilitation adjustment device according to the embodiment of the present invention; and

FIG. 6 is a back view of the cervical spine rehabilitation adjustment device according to the embodiment of the present invention.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

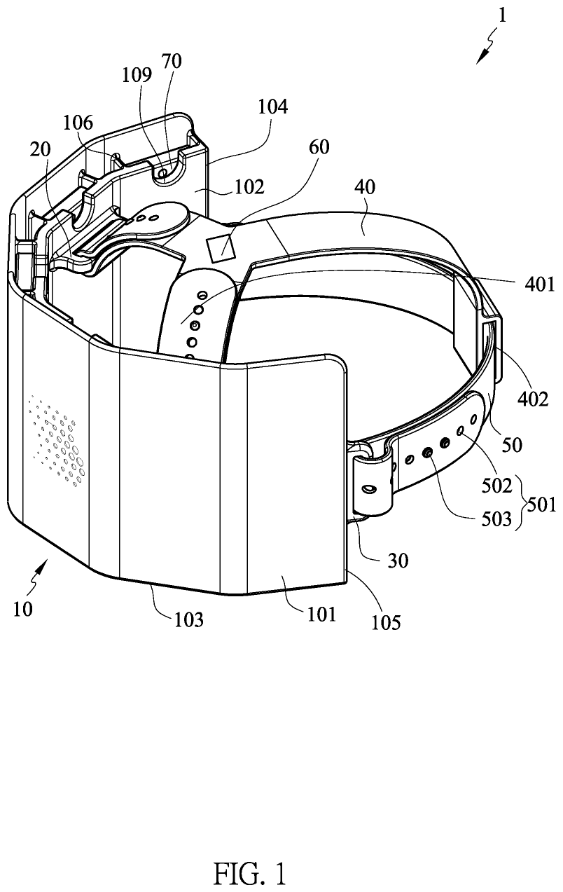

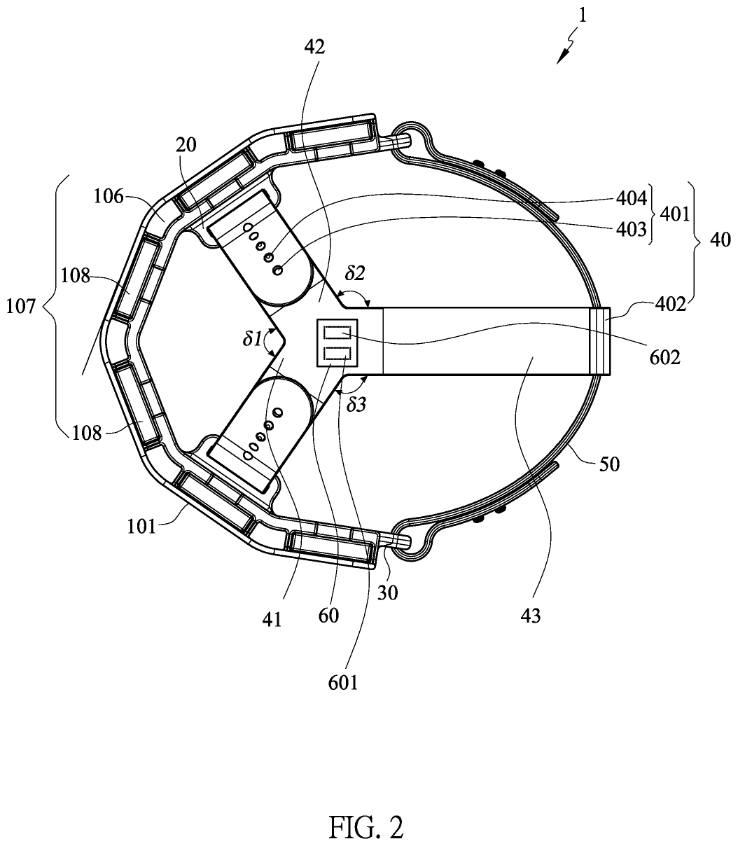

Please refer to FIG. 1 and FIG. 2, FIG. 1 is a stereoscopical schematic view of a vertebrae adjustment device according to an embodiment of the present invention; and FIG. 2 is a top view of the cervical spine rehabilitation adjustment device according to the embodiment of the present invention.

As shown in FIG. 1 and FIG. 2, the present invention provides a cervical spine rehabilitation adjustment device 1 comprising a weight pocket 10, two fixing rings 20, two connection rings 30, a first fixing belt 40, a second fixing belt 50 and a sensing device 60; wherein the weight pocket 10 having a front plate 101, a rear plate 102, a bottom plate 103, a right side plate 104, a left side plate 105 and a plurality of connecting parts 106. Further, the front plate 101 and the rear plate 102 are connected to each other by the right side plate 104, the left side plate 105 and the bottom plate 103 to form an accommodating space 107, and the connecting parts 106 are mounted in the accommodating space 107 to divide the accommodating space 107 into a plurality of separations 108. Furthermore, a plurality of weight blocks 70 arranged into the separations 108, and a plurality of notches 109 disposed on the upper edge of the rear plate 102; wherein the weight blocks 70 can be easily taken out or placed in the separations 108 by the notches 109. Secondly, the two fixing rings 20 mounted on the rear plate 102; and two connection rings 30 respectively mounted on the right side plate 104 and the left side plate 105.

As shown in FIG. 2, the first fixing belt 40 has two first fixing sections 401 and a through hole 402; wherein the two first fixing sections 401 are respectively disposed on the front ends of the two bifurcated extensions of the first fixing belt 40, and the two bifurcated extensions of the first fixing belt 40 are fixed on the two first fixing rings 20 respectively by the two first fixing sections 401, and the through hole 402 is provided on the rear end of the first fixing belt 40. Each of the two first fixing sections 401 is composed of a first fastener 403 and a second fastener 404, and the first fastener 403 is correspondingly fastened to the second fastener 404. As shown in FIG. 1, the second fixing belt 50 has two second fixing portions 501 respectively disposed on both ends of the second fixing belt 50, and the second fixing belt 50 passes through the through hole 402 and the second fixing belt 50 is fixed on the two connecting rings 30 by the two second fixing portions 501; wherein each of the two second fixing portions 501 is composed of a third fastener 502 and a fourth fastener 503, and the third fastener 502 is correspondingly fastened to the fourth fastener 503. In addition, the first fastener 403 and the second fastener 404 are sequentially disposed on the front ends of the first fixing belt 40; and the third fastener 502 and the fourth fastener 503 are disposed on the front ends of the second fixing belt 50. There is a gap between the first fastener 403 and the second fastener 404; alternatively, there is another gap between the third fastener 502 and the fourth fastener 503. The tightness of the first fixing belt 40 and the second fixing belt 50 are adjusted by the first fastener 403 or the third fastener 502 fastened to the second fastener 404 or the fourth fastener 503.

Additionally, the sensor 60 is movably disposed on the vertebrae adjustment device 1 through a fastener 60, and the sensor 60 comprises a sensing unit 601 and a processing unit 602 electrically connected to the sensing unit 601; wherein the sensor 60 senses movements of the head, neck or spine of a human body, and outputs a sensing data indicating the activity state of the human body. Further, the processing unit 602 generates a posture data of a cervical angle or a spine angle of the human body according to the sensing data, and outputs a warning notification according to the posture data. The processing unit 602 produces the warning notification when the processing unit 602 judges that a warning condition is met according to the posture data; and the warning condition includes that a first cumulative time of the human body's cervical spine angle outside a first angle reaches a first threshold, or a second cumulative time of the human body's spine angle outside a second angle reaches a second threshold. In addition, an installation location of the sensor 60 is not particularly limited, and can be placed on any location based on the needs of the user. If the user wants to sense the movement position of the human head, the sensor 60 can be placed on the first fixing belt 40 or the second fixing belt 50; if the movement position of the neck is to be sensed, the sensor 60 can be placed on the human neck; and if the movement position of the human spine is to be sensed, the sensor 60 can be placed on the human spine, but the present invention is not limited to thereon.

In addition, the materials of the weight pocket 10, the two first fixing rings 20, the two connecting rings 30, the first fixing belt 40 and the second fixing belt 50 may be randomly varied based on the user's requirements. Further, the material of the weight pocket 10, the two first fixing rings 20, the two connecting rings 30, the first fixing belt 40 and the second fixing belt 50 may be composed of the same material or different materials, and the material may be a non-absorbent material to prevent mildew caused by sweating; wherein the non-absorbent material may be a polymer material, such as, silicone, polyvinyl chloride, etc. In a preferred embodiment, the weight pocket 10, the two first fixing rings 20 and the two connecting rings 30 may be made of silicone material, and the first fixing belt 40 and the second fixing belt 50 are made of polyvinyl chloride material, but the present invention is not limited thereto.

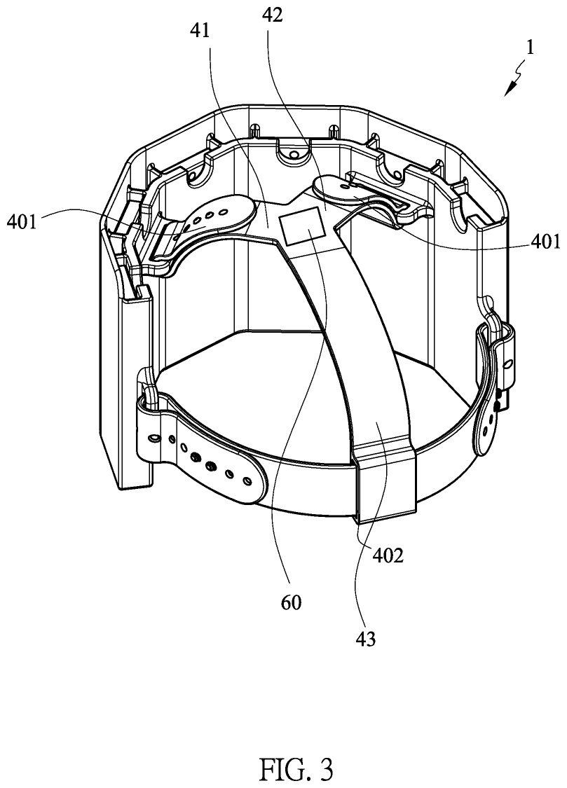

Please refer to FIG. 3, FIG. 3 is a back view of a rear view of the cervical spine rehabilitation adjustment device according to the embodiment of the present invention.

As shown in FIG. 3, the first fixing belt 40 is composed of a first branch belt 41, a second branch belt 42 and a main body belt 43. The two first fixing sections 401 are respectively disposed on the front end of the first branch belt 41 and the second branch the belt 42, and the through hole 402 is provided on the rear end of the main body belt 43. Further, the rear end of the first branch belt 41 and the rear end of the second branch belt 42 are connected to the front end of the main body belt 43.

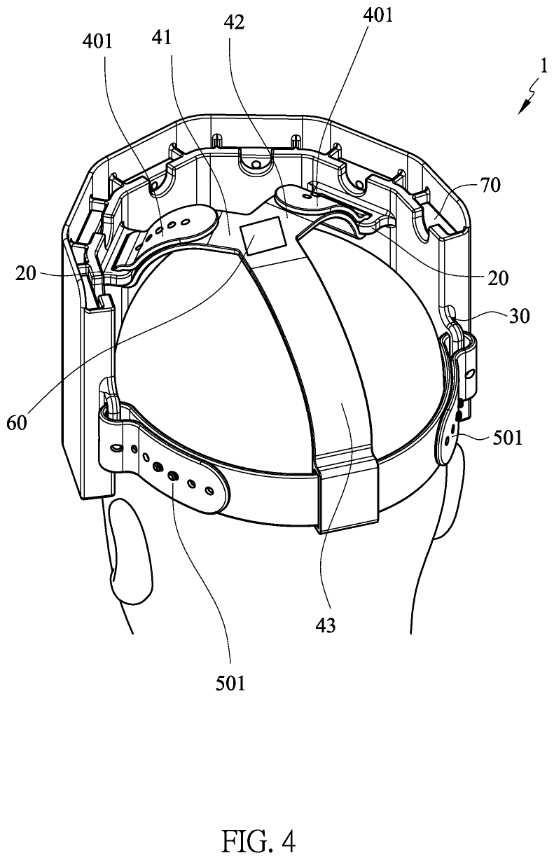

Please refer to FIG. 4. FIG. 4 is a schematic view of the cervical spine rehabilitation adjustment device when worn, according to the embodiment of the present invention.

As shown in FIG. 4, firstly, when the patient uses the cervical spine rehabilitation adjustment device 1 of the present invention, the patient's neck and spine are taken with X-rays to produce an X-ray film, and a doctor judges the number and weight of the weight blocks 70 used by the X-ray film Furthermore, when the patient uses the cervical spine rehabilitation adjustment device 1 of the present invention, the patient placed the rear plate 102 of the weight pocket 10 on the forehead, and then the first branch belt 41 and the second branch belt 42 of the first fixing belt 40 pass through the two first fixing rings 20 respectively and fastened to the two first fixing sections 401. Secondly, both ends of the second fixing belt 50 pass through the two connecting rings 30 and fastened to the two second fixing portions 501 to fix the vertebrae adjustment device 1 on the user's head, and the weight blocks 70 are loaded into the separations 108 to correct the cervical vertebrae.

As shown in FIG. 2, the first branch belt 41 and the second branch belt 42 form a first included angle δ1, and the first included angle δ1 is 30 to 180 degrees. In addition, when the first included angle δ1 is less than 180 degrees, the first branch belt 41 and the second branch belt 42 form a Y-shaped structure; and when the first included angle δ1 is 180 degrees, the first brand belt 41 and the second branch belt 42 form a T-shaped structure. Further, the first branch belt 41 and the main body belt 43 form a second included angle δ2; the second branch belt 42 and the main body belt 43 form a third included angle δ3; and the second included angle δ2 is equal to the third included angle δ3. When the main body belt 43 reaches the top of the head, the first brand belt 41 and the second branch belt 42 will not reach the forehead, due to the angles of the second included angle δ2 and the third included angle δ3 and the length of the first branch belt 41, the second branch belt 42 and the main body belt 43.

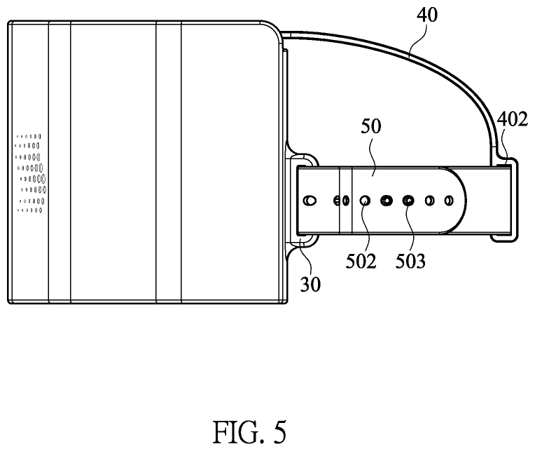

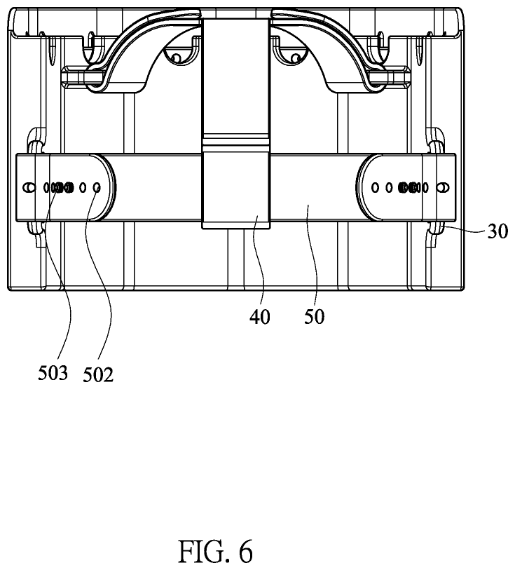

Please refer to FIG. 5 and FIG. 6, FIG. 5 is a side view of the vertebrae adjustment device according to the embodiment of the present invention; and FIG. 6 is a back view of the vertebrae adjustment device according to the embodiment of the present invention.

As shown in FIG. 5 and FIG. 6, the third fastener 502 and the fourth fastener 503 are provided on the front end of the second fixing belt 50; and there is a gap between the third fastener 502 and the fourth fastener 503. The second fixing belt 50 passes through the two connecting rings 30, and the length of the second fixing belt 50 is adjusted by fastening the third fastener 502 to the fourth fastener 503. Furthermore, as shown in FIG. 5, the first fixing belt 40 has the through hole 402, the second fixing belt 50 passes through the through hole 402, so that the second fixing belt 50 is movably connected to the first fixing belt 40. Therefore, the patient can adjust the relative position of the first fixing belt 40 and the second fixing belt by the through hole 402. As shown in FIG. 6, the fourth fastener 503 is a cylinder with a threaded structure on its surface; and the fourth fastener 503 is fastened to the third fastener 502 by the threaded structure, so that the contact area between the fourth fastener 503 and the third fastener 502 generates friction to increase the fastening ability.

The foregoing description, for the purpose of explanation, has been described with reference to specific embodiments; however. The embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the disclosure to the precise forms disclosed. Modifications and variations are possible in view of the above demonstrations.

I claim:

1. A vertebrae adjustment device, comprising:
a weight pocket having a front plate, a rear plate, a bottom plate, a right side plate, a left side plate and a plurality of connecting parts; wherein the front plate and the rear plate are connected to each other by the right side plate, the left side plate and the bottom plate to form an accommodating space, and the connecting parts are mounted in the accommodating space to divide the accommodating space into a plurality of separations;
two fixing rings mounted on the rear plate;
two connection rings respectively mounted on the right side plate and the left side plate;
a first fixing belt having two first fixing sections and a through hole; wherein the two first fixing sections are respectively disposed at the front ends of the two bifurcated extensions of the first fixing belt, the two bifurcated extensions of the first fixing belt are fixed on the two first fixing rings respectively by the two first fixing sections; and the through hole is provided on the rear end of the first fixing belt; and
a second fixing belt having two second fixing sections respectively disposed on both ends of the second fixing belt, and the second fixing belt passes through the through hole and fixed on the two connecting rings by the two second fixing sections;
a sensor movably disposed on the cervical spine rehabilitation adjustment device through a fastener, the sensor comprising a sensing unit and a processing unit electrically connected to the sensing unit;
wherein the sensor senses movements of the head, neck or spine of a human body, and outputs a sensing data indicating the activity state of the human body;
wherein the processing unit generates a posture data of a cervical angle or a spine angle of the human body according to the sensing data, and outputs a warning notification according to the posture data.

2. The cervical spine rehabilitation adjustment device in claim 1, further comprising a plurality of weight blocks loaded into the separations.

3. The vertebrae adjustment device in claim 1, further comprising a plurality of notches disposed on the upper edge of the rear plate.

4. The cervical spine rehabilitation adjustment device in claim 1, wherein the two first fixing sections and the two second fixing section are respectively composed of a male fastener and a female fastener, and the male fastener is correspondingly fastened to the female fastener.

5. The cervical spine rehabilitation adjustment device in claim 4, wherein male fastener and the female fastener are arranged on the front ends of the first fixing belt and the second fixing belt, with a distance between the male fastener and the female fastener.

6. The cervical spine rehabilitation adjustment device in claim 4, wherein the two first fixing sections and the two second fixing sections are respectively composed of a sticky part and a bonding part corresponding to the sticky part, and the sticky part is correspondingly bonded to the bonding part.

7. The cervical spine rehabilitation adjustment device in claim 1, wherein the first fixing belt is composed of a first branch belt, a second branch belt and a main body belt; the two first fixing sections are respectively disposed on the front ends of the first branch belt and the second branch belt; the through hole is provided on the rear end of the main body belt; and the rear end of the first branch belt and the rear end of the second branch belt are connected to the front end of the main body belt.

8. The cervical spine rehabilitation adjustment device in claim 6, wherein the first branch belt and the second branch belt form a first included angle, and the first included angle is 30˜180 degrees.

9. The cervical spine rehabilitation adjustment device in claim 6, wherein the first branch belt and the main body belt form a second included angle, the second branch belt and the main body belt form a third included angle, and the second included angle is equal to the third included angle.
